(19) United States
(12) Patent Application Publication
(10) Pub. No.: US 2009/0246776 A1
(43) Pub. Date: Oct. 1, 2009

(54) Modulators of scarb-1 for treating acne or hyperseborrhea

(75) Inventors: Fernand Labrie, Quebec (CA); Isabelle Carlavan, Grasse (FR); Pascal Collette, Le Cannet (FR); Ezequiel L. Calvo, Quebec (CA); Jerome Aubert, Grasse (FR); Johannes Voegel, Chateauneuf/Grasse (FR)

Correspondence Address: BUCHANAN, INGERSOLL & ROONEY PC, POST OFFICE BOX 1404, ALEXANDRIA, VA, 22313-1404, US

(73) Assignee: GALDERMA RESEARCH & DEVELOPMENT, Biot (FR)

(21) Appl. No.: 12/320,166

(22) Filed: Jan. 21, 2009

Related U.S. Application Data
(63) Continuation of application No. PCT/FR2007/051686, filed on Jul. 18, 2007.

(30) Foreign Application Priority Data
Jul. 19, 2006 (FR) 0653034

(51) Int. Cl. C12Q 1/68

(52) U.S. Cl. 435/6

(57) ABSTRACT

An in vitro method for screening candidate compounds for the preventive or curative treatment of acne, includes the determination of the capacity of a compound to modulate the expression or the activity of the SCARB-1 receptor, and the use of modulators of the expression or activity of this receptor for the treatment of acne or skin disorders associated with a hyperseborrhea; methods for the in vitro diagnosis or prognosis of these pathologies are also described.

### CROSS-REFERENCE TO PRIORITY/PCT APPLICATIONS

This application claims priority under 35 U.S.C. § 119 of FR 0653034, filed Jul. 19, 2006, and is a continuation/national phase of PCT/FR 2007/051686, filed Jul. 18, 2007, and designating the United States (published in the French language on Jan. 24, 2008 as WO 2008/009859 A2; the title and abstract were also published in English), each hereby expressly incorporated by reference in its entirety and each assigned to the assignee hereof.

### BACKGROUND OF THE INVENTION

1. Technical Field of the Invention

The present invention relates to the identification and administration of the SCARB-1 receptor modulating compounds for the treatment of acne and skin disorders associated with a hyperseborrhea. This invention also relates to methods for the in vitro diagnosis or in vitro prognosis of these pathologies.

2. Description of Background and/or Related and/or Prior Art

A hyperseborrheic greasy skin is characterized by excessive secretion and excretion of sebum. Conventionally, a sebum level greater than 200 μg/cm², measured in the region of the forehead, is considered as being characteristic of a greasy skin. A greasy skin is often associated with a desquamation defect, a glistening complexion and a thick skin grain. In addition to these aesthetic disorders, excess sebum can serve as a support for the anarchical development of saprophytic bacterial flora (P. acnes in particular), and cause the appearance of comedones and/or acne lesions.

This stimulation of the production of sebaceous glands is induced by androgens. Acne is in fact a chronic disease of the pilosebaceous follicle under hormonal control. A hormone therapy against acne is one possibility of treatment for women, the aim being to prevent the effects of androgens on the sebaceous gland. In this context, therapy is generally made of oestrogens, anti-androgens or agents reducing the production of androgens by the ovaries or the adrenal gland. The anti-androgens administered for the treatment of acne include in particular spironolactone, cyproterone acetate and flutamide. However, these agents have severe side effects. Thus, any pregnancy must be absolutely prevented, in particular because of a risk of feminization for the male fetus. These agents are banned in male patients.

Need therefore exists to identify mediators downstream of the action of the steroid hormones and to modulate them in order to provide a similar therapeutic profile, but with reduced side effects.

### SUMMARY OF THE INVENTION

It has now been discovered that the gene encoding SCARB-1 receptor was expressed in the human sebaceous glands, and that its expression was regulated by androgens, in vivo, in a mouse preputial gland model. Thus, targeting the SCARB-1 gene or its expression product is now proposed to prevent or improve acne phenomena and skin disorders associated with a hyperseborrhea, in particular the appearance of greasy skin.

The expression acne means all the forms of acne, namely, in particular acne vulgaris, comedo type acne, polymorphic acne, nodulocystic acne, acne conglobata, or secondary acnes such as solar acne, acne medicamentosa or occupational acne.

This invention also provides in vitro diagnostic or in vitro prognostic methods based on the detection of the level of expression or activity of SCARB-1.

### BRIEF DESCRIPTION OF THE DRAWINGS

FIG. 1 shows the measurement of the expression of the SCARB-1 gene in gonadectomized male mice treated with various vehicles, and

FIG. 2 is a graph of a kinetic study of 15 to 96 hours.

### DETAILED DESCRIPTION OF BEST MODE AND SPECIFIC/PREFERRED EMBODIMENTS OF THE INVENTION

SCARB-1:

Scavenger receptors are cell surface proteins which are typically found on macrophages and which bind to various types of modified lipoproteins such as low-density lipoproteins (LDL). This family of transmembrane receptors, which exhibits great structural diversity, is involved in endocytosis and phagocytosis of apoptotic cells and bacteria, and in cell adhesion. These receptors are also thought to be involved in the deposition of the LDL cholesterol of macrophages at the level of the walls of the coronary arteries during the first phases of the formulation of an atherosclerotic plaque.

SCARB-1 (scavenger receptor class B, member 1, also called SRB-1, or Cla-1) belongs to the family of scavenger receptors class B, and is a receptor for the high-density lipoproteins, which mediates the incorporation of cholesterol into cells. SRB-1 can also serve as a receptor for the non-HDL lipoproteins and is thought to play an important role in the reverse transport of cholesterol. The expression of this gene is thought to be induced by testosterone in macrophages and hepatocytes in culture (Langer et al., Biochem Biophys Res Commun., 2002, 296(5):1051-1057). Millena et al. (Mol Cell Endocrinol., 224(1-2):29-39) have, for their part, shown that an increase in androgen production in response to TGFβ was associated with high levels of mRNA for SCARB-1.

In the context of the present invention, the term “SCARB-1 gene” or “SCARB-1 nucleic acid” means the gene or nucleic sequence encoding a transmembrane SCARB-1 receptor. If the intended target is preferably the human gene or its expression product, the invention may also call into play cells expressing a heterologous SCARB-1 receptor, through genomic integration or transient expression of an exogenous nucleic acid sequence encoding the enzyme (for example, an enzyme from another organism).

A human cDNA sequence for SCARB-1 is reproduced in the annex (SEQ ID No. 1). It is the sequence NM005505 whose coding moiety is located from nucleic acid 70 to 1599.

Diagnostic Applications:

The present invention features an in vitro method for the diagnosis or monitoring of the progression of acne lesions or of a skin disorder associated with a hyperseborrhea in a subject, comprising comparing the expression or the activity of the SCARB-1, the expression of its gene or the activity of at least one of its promoters, in a biological sample from a subject compared with a biological sample from a control subject.

The expression of the protein may be determined by an assay of the SCARB-1 protein by radioimmunoassay, for example by ELISA assay. Another method, in particular for measuring the expression of the gene, is to measure the quantity of corresponding mRNA, by any method as described above. An assay of the activity of SCARB-1 may also be employed.

In the context of a diagnosis, the “control” subject is a “healthy” subject.

In the context of a monitoring of the progression of acne lesions or of a skin disorder linked to a hyperseborrhea, the “control subject” refers to the same subject at a different time, which preferably corresponds to the start of the treatment (To). This measurement of the difference in the expression or in the activity of SCARB-1, or the expression of its gene or the activity of at least one of its promoters, makes it possible in particular to monitor the efficacy of a treatment, in particular a treatment with an SCARB-1 modulator, as indicated above, or with another treatment against acne or a skin disorder associated with a hyperseborrhea. Such a monitoring can reassure the patient regarding the justification or the need for pursuing this treatment.

The present invention also features an in vitro method for determining the predisposition of a subject to develop acne lesions or a skin disorder associated with a hyperseborrhea, comprising comparing the expression or the activity of the SCARB-1, the expression of its gene or the activity of at least one of its promoters, in a biological sample from a subject compared with a biological sample from a control subject.

Here again, the expression of the protein may be determined by an assay of the SCARB-1 protein by radioimmunoassay, for example by ELISA assay. Another method, in particular for measuring the expression of the gene, is to measure the quantity of corresponding mRNA by any method as described above. An assay of the activity of SCARB-1 may also be employed.

The subject tested is here an asymptomatic subject with no skin disorder linked to a hyperseborrhea or an acne. The “control” subject in this method means a “healthy” reference subject or population. The detection of this predisposition allows the putting in place of a preventive treatment and/or an increased monitoring of the signs linked to acne or to a skin disorder associated with a hyperseborrhea.

In these in vitro diagnostic or prognostic methods, the biological test sample may be any biological fluid sample or a sample of a biopsy. Preferably, the sample may, nevertheless, be a preparation of skin cells obtained for example by desquamation or biopsy. It may also be sebum.

Screening Methods:

This invention also features an in vitro method for screening candidate compounds for the preventive and/or curative treatment of acne, or of the skin disorders associated with a hyperseborrhea, comprising determining the capacity of a compound to modulate the expression or activity of the SCARB-1 receptor or the expression of its gene or the activity of at least one of its promoters, the said modulation indicating the usefulness of the compound for the preventive or curative treatment of acne or of the skin disorders associated with a hyperseborrhea. The method therefore makes it possible to select the compounds capable of modulating the expression or activity of the SCARB-1 receptors, or the expression of its gene or the activity of at least one of its promoters.

More particularly, this invention features an in vitro method for screening candidate compounds for the preventive and/or curative treatment of acne or skin disorders associated with a hyperseborrhea, comprising the following steps:

a. preparing at least two biological samples or reaction mixtures;

b. bringing one of the samples or reaction mixtures into contact with one or more test compounds;

c. measuring the expression or activity of the protein SCARB-1, the expression of its gene or the activity of at least one of its promoters, in biological samples or reaction mixtures;

d. selecting the compounds for which a modulation of the expression or activity of the protein SCARB-1, the expression of its gene or the activity of at least one of its promoters, is measured in the sample or mixture treated in b), compared with the untreated sample or mixture.

The expression “modulation” means any effect on the expression or activity of the receptor, the expression of its gene or the activity of at least one of its promoters, namely, optionally a partial or complete stimulation (i.e., an agonist activity), but preferably a partial or complete inhibition (i.e., an antagonist activity). The difference in expression obtained with the test compound compared with a control prepared in the absence of the compound is significant from 25% or more.

In the present text, unless otherwise specified, “expression of a protein” means the quantity of this protein.

The expression “activity of a protein” means its biological activity.

The expression “activity of a promoter” means the capacity of this promoter to trigger the transcription of the DNA sequence coded downstream of this promoter (and therefore indirectly the synthesis of the corresponding protein).

The test compounds may be of any type. They may be of a natural origin or may have been produced by chemical synthesis. This may be a library of structurally defined chemical compounds, non-characterized compounds or substances or a mixture of compounds.

Various techniques may be used to test these compounds and identify the compounds of therapeutic interest, modulators of the expression or the activity of the SCARB-1 receptor.

According to a first embodiment, the biological samples are cells transfected with a reporter gene that is operably linked to all or part of the promoter of the SCARB-1 gene, and step c) described above consists in measuring the expression of the said reporter gene.

The reporter gene may in particular encode an enzyme which, in the presence of a given substrate, leads to the formation of colored products, such as CAT (chloramphenicol acetyltransferase), GAL (beta-galactosidase) or GUS (beta-glucuronidase). This may also be the luciferase gene or GFP (Green Fluorescent Protein). The assay of the protein encoded by the reporter gene, or its activity, is carried out in a conventional manner by calorimetric, fluorometric or chemiluminescent techniques, among others.

According to a second embodiment, the biological samples are cells expressing the SCARB-1 gene, and step c) described above entails measuring the expression of the said gene.

The cell employed here may be of any type. This may be a cell endogenously expressing the SCARB-1 gene, such as, for example, a liver cell, an ovarian cell or even better a sebocyte. It is also possible to employ organs of human or animal origin, such as for example the preputial gland, clitorial gland or sebaceous gland of the skin.

This may also be a cell transformed with a heterologous nucleic acid encoding a SCARB-1 receptor, preferably of human origin, or of mammalian origin.

A wide variety of host cell systems may be used, such as, for example, Cos-7, CHO, BHK, 3T3, HEK293 cells.

The nucleic acid may be stably or transiently transfected by any method known to one skilled in this art, for example using calcium phosphate, DEAE-dextran, liposome, viruses, electroporation or microinjection.

In this method, the expression of SCARB-1 may be determined by measuring the level of transcription of the said gene, or its level of translation.

The expression level of transcription of a gene means the quantity of corresponding mRNA produced. The expression level of translation of a gene means the quantity of corresponding protein produced.

One skilled in this art is familiar with techniques allowing the quantitative or semi-quantitative detection of the mRNA of a gene of interest. The techniques based on the hybridization of mRNA with specific nucleotide probes are the most common (Northern Blot, RT-PCR, protection using RNase). It may be advantageous to employ detection markers such as fluorescent, radioactive or enzymatic agents or other ligands (for example avidin/biotin).

In particular, the expression of the gene may be measured by real-time PCR or by protection using RNase. The expression protection using RNase means the detection of a known mRNA among poly(A) RNAs of a tissue, which may be carried out with the aid of a specific hybridization with a labeled probe. The probe is a labeled (radioactive) complementary RNA for the messenger to be detected. It may be constructed from a known mRNA whose cDNA, after RT-PCR, has been cloned into a phage. The poly(A) RNA of the tissue where the sequence is to be detected is incubated with this probe under slow hybridization conditions in liquid medium. RNA:RNA hybrids are formed from the mRNA to be detected and the anti-sense probe. The hybridized medium is then incubated with a mixture of ribonucleases specific for single-stranded RNA, such that only the hybrids formed with the probe can withstand this digestion. The product of digestion is then deproteinized and repurified before being analyzed by electrophoresis. The labeled hybridized RNAs are detected by autoradiography.

The level of translation of the gene is evaluated for example by immunological assay of the product of the said gene. The antibodies employed for this effect may be of the polyclonal or monoclonal type. Their production involves conventional techniques. An anti-SCARB-1 polyclonal antibody may, inter alia, be obtained by immunization of an animal such as a rabbit or a mouse, with the whole enzyme, collection and then depletion of the anti-serum according to methods known per se by one skilled in this art. A monoclonal antibody may, inter alia, be obtained by the conventional Kôhler and Milstein method (Nature (London), 256: 495-497 (1975)). Other methods of preparation of monoclonal antibodies are also known. It is possible, for example, to produce monoclonal antibodies by expressing a nucleic acid cloned from a hybridoma. It is also possible to produce antibodies by the phage display technique by introducing antibody cDNAs into vectors, which are typically filamentous phages which display V gene libraries at the surface of the phage (for example fUSE5 for E. coli).

The immunological assay may be carried out in a solid phase or in a homogeneous phase; in a single stage or in two stages; as a sandwich method or as a competitive method, by way of non-limiting examples. According to a preferred embodiment, the capture antibody is immobilized on a solid phase. It is possible to employ, by way of non-limiting examples of a solid phase, microplates, in particular polystyrene microplates, or solid particles or beads, paramagnetic beads.

ELISA assays, radio-immunoassays or any other detection technique may be carried out in order to reveal the presence of the antigen-antibody complexes formed.

The characterization of the antigen-antibody complexes, and more generally of the isolated or purified proteins, but also recombinant proteins (obtained in vitro and in vivo), may be carried out by mass spectrometry analysis. This identification is made possible by virtue of the analysis (determination of the mass) of peptides generated by the enzymatic hydrolysis of the proteins (trypsin in general). Generally, the proteins are isolated according to methods known to one skilled in this art, prior to the enzymatic digestion. The analysis of the peptides (in hydrolysate form) is performed by separation of the peptides by HPLC (nano-HPLC) based on their physicochemical properties (reversed phase). The determination of the mass of the peptides thus separated is carried out by ionization of the peptides or by direct coupling to mass spectrometry (electrospray ESI mode) or after deposition and crystallization in the presence of a matrix known to one skilled in this art (analysis in MALDI mode). The proteins are then identified using appropriate software (for example Mascot).

According to a third embodiment, step a) described above entails preparing reaction mixtures each comprising an SCARB-1 receptor and step c) described above entails measuring the capacity of the compound to bind to activity.

The SCARB-1 receptor may be produced according to customary techniques using Cos-7, CHO, BHK, 3T3 and HEK293 cells. It may also be produced with the aid of microorganisms such as bacteria (for example E. coli or B. subtilis), yeasts (for example Saccharomyces, Pichia) or insect cells, such as Sf9 or Sf21.

Measurement of activity refers to the determination of the cell adhesion activity, transporter activity, apoptotic activity, activity in lipid metabolism, in signal transduction and/or in cytokine secretion.

An example of a method of detection is presented in TIAN et al., Bio Env Sci., 2005, 18:265-272. This method of detection of the activity of the receptor is based on the use of fluorescence-labeled human lipoproteins, Dil-lipoproteins (Dil-AcLDL and DiLHDL). The measurement of the binding of Dil-lipoproteins is carried out by incubation of Sf9 cells, transformed beforehand with a baculovirus at an M.O.I (multiplicity of infection) of 5 with baculoviruses possessing or not possessing the coding sequence for the SCARB1 receptor, in 50 μL of Grace's culture medium containing 0.2% BSA (bovine serum albumin) with various concentrations of Dil-AcLDL and Dil-HDL at room temperature for a few hours. After incubation, the cells were washed twice with a cold phosphate buffer (PBS), detached with a pipette and transferred onto a plate containing 100 μL of PBS. The results were quantified with a fluorescence plate reader. The specific binding activity of the receptor is measured by calculating the difference from the binding activity of cells transformed with the baculovirus not expressing the receptor (control) and the binding activity of cells transformed with the baculovirus expressing the receptor.

Modulators of the Receptor:

This invention also features the use of a modulator of the human receptor SCARB-1 which can be obtained by one of the above methods, for the preparation of a medicament useful for the preventive and/or curative treatment of acne, or of skin disorders associated with a hyperseborrhea.

A method for the preventive and/or curative treatment of acne, or of skin disorders associated with a hyperseborrhea, is thus described here, the regime or regimen comprising the administration of a therapeutically effective quantity of a modulator of the human receptor SCARB-1, to a patient requiring such a treatment.

This invention also features the cosmetic application of a modulator of the SCARB-1 receptor for the aesthetic treatment of greasy skins.

Preferably, the modulator is an antagonist of the receptor. The term “antagonist” or “inhibitor” refers to a chemical compound or substance which substantially eliminates or reduces the activity of the SCARB-1 receptor. The term “substantially” means a reduction of at least 25%, preferably of at least 35%, preferably still of at least 50%, and more preferably of at least 70% or 90%. More particularly, it may be a compound which interacts with, and blocks, the binding from the receptor and its ligand.

A preferred inhibitor interacts with the receptor in solution at inhibitor concentrations of less than 1 μM, preferably of less than 0.1 μM, preferably still of less than 0.01 μM.

The modulator compound may also be a polypeptide, a DNA or RNA anti-sense polynucleotide, an si-RNA or a PNA (“peptide nucleic acid”, polypeptide chain substituted with purine and pyrimidine bases whose spatial structure mimics that of DNA and allows hybridization thereto).

The modulator compound may be an anti-SCARB-1 receptor inhibitory antibody, preferably a monoclonal antibody. Advantageously, such an inhibitory antibody is administered in a quantity sufficient to obtain a plasma concentration of about 0.01 μg per ml to about 100 μg/ml, preferably of about 1 μg per ml to about 5 μg/ml.

It may be for example an antibody which binds to the extracellular collagen domain of the receptor, which plays a role in the binding to the ligand (Acton, 1993, J. Biol. Chem., 268 (5): 3530-7). Examples of neutralizing antibodies are also described in WO 04/80385. Other neutralizing antibodies are known in the prior art (see, for example, Frolov, 2000, J. Biol. Chem., 275(17): 12769-12780).

The invention comprises the administration of such SCARB-1 receptor antagonist compounds for the preventive and/or curative treatment of acne or any skin disorder associated with a hyperseborrhea.

Other modulator compounds identified by the screening method described above are also useful.

The modulator compounds are formulated in a pharmaceutical composition, in combination with a pharmaceutically acceptable vehicle. These compositions may be administered for example orally, parenterally or topically. Preferably, the pharmaceutical composition is applied topically. By the oral route, the pharmaceutical composition may be provided in the form of tablets, gelatin capsules, sugar-coated tablets, syrups, suspensions, solutions, powders, granules, emulsions, suspensions of microspheres or nanospheres or lipid or polymer vesicles allowing controlled release. By the parenteral route, the pharmaceutical composition may be provided in the form of solutions or suspensions for infusion or injection.

By the topical route, the pharmaceutical composition is more particularly useful for the treatment of the skin and the mucous membranes and may be provided in the form of salves, creams, milks, ointments, powders, impregnated pads, solutions, gels, sprays, lotions or suspensions. It may also be provided in the form of suspensions of microspheres or nanospheres or of lipid or polymer vesicles or of polymer patches or hydrogels allowing controlled release. This composition for topical application may be provided in anhydrous form, in aqueous form or in the form of an emulsion. In a preferred embodiment, the pharmaceutical composition is provided in the form of a gel, a cream or a lotion.

The composition may comprise an amount of SCARB modulator ranging from 0.001 to 10% by weight, in particular from 0.01 to 5% by weight relative to the total weight of the composition.

The pharmaceutical composition may additionally contain inert additives or combinations of these additives, such as:
wetting agents;
taste enhancing agents;
preservatives such as para-hydroxybenzoic acid esters;
stabilizing agents;
moisture regulating agents;
pH regulating agents;
osmotic pressure modifying agents;
emulsifying agents;
UV-A and UV-B screening agents;
   and antioxidants, such as alpha-tocopherol, butylated hydroxyanisole or butylated hydroxytoluene, Super Oxide Dismutase, Ubiquinol or certain metal chelators.

Legend for the Figures:

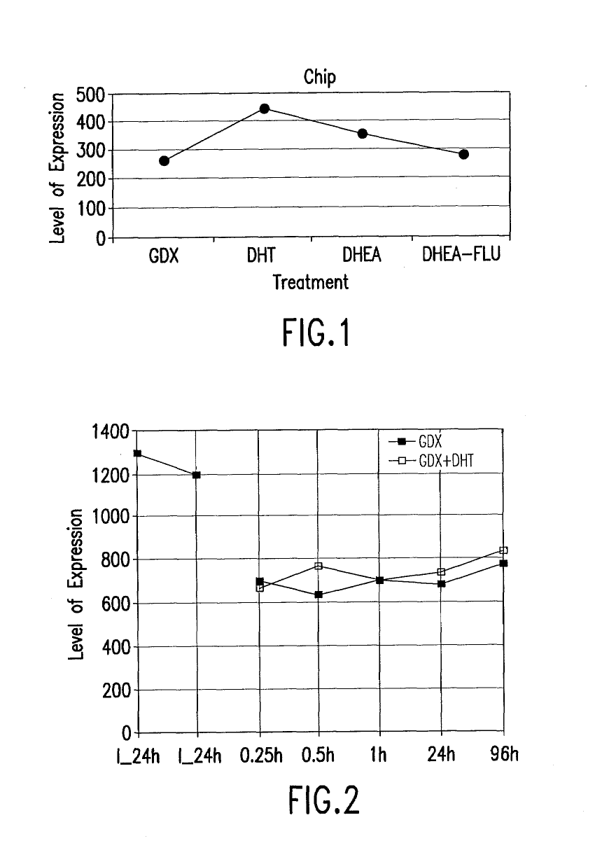

FIG. 1 shows the measurement of the expression of the SCARB-1 gene in gonadectomized male mice treated with the vehicle, DHT, DHEA or the combination of DHEA-Flutamide for a period of 7 days once per day (long-term treatment). After the experiment was finished, the preputial glands were removed, the RNA was isolated and the expression of the genes was analyzed by the Affymetrix technique.

GDX: gonadectomized mice treated with the vehicle

DHT: gonadectomized mice treated with Dihydrotestosterone (agonist of the androgen receptor)

DHEA: gonadectomized mice treated with Dihydroepiandrosterone (precursor of the steroid hormones; in the preputial glands metabolized to the active androgen)

DHEA-Flu: gonadectomized mice treated with a combination of Dihydroepiandrosterone and Flutamide (antagonists of the androgen receptor; which blocks the effects of the DHT and DHEA agonists).

Level of expression: level of expression of the mRNA

FIG. 2 is a graph of a kinetic study of 15 to 96 hours. Points 1_{—}24h and 1_{—}24h(R1) show the level of expression of SCARB-1 of control mice (=non-gonadectomized mice; duplicate) at the 24 hour point. The next points are from gonadectomized mice and indicate the successive times (in hours) of the kinetic study.

The blue dotted line is the expression in the gonadectomized mice following treatment with DHT at the time zero. The red line is the expression in gonadectomized mice without DHT treatment.

Level of expression: level of expression of the mRNA

In order to further illustrate the present invention and the advantages thereof, the following specific examples are given, it being understood that same are intended only as illustrative and in nowise limitative. In said examples to follow, all parts and percentages are given by weight, unless otherwise indicated.

### EXAMPLES

### Experimental Data

### Example 1

### Expression of SCARB-1 in the human sebaceous gland and in the human epidermis

Human sebaceous glands were separated from the human epidermis by treatment with dispase and dissection under a binocular lens. Samples of total RNA were prepared from the sebaceous glands and from the epidermis.

The expression of the genes was analyzed on an Affymetrix station (microfluidic model; hybridization oven; scanner; computer) following the protocols provided by the company. Briefly, the total RNA isolated from the tissues is transcribed to cDNA. From the double-stranded cDNA, a cRNA labeled with biotin is synthesized using T7 polymerase and a precursor NTP conjugated to biotin. The cRNAs are then fragmented to small sized fragments. All the molecular biology steps are checked using the Agilent “Lab on a chip” system in order to confirm the good efficiencies of the enzymatic reactions. The Affymetrix chip is hybridized with the biotinylated cRNA, rinsed and then fluorescence labeled using a fluorophore conjugated to streptavidin. After washings, the chip is scanned and the results are calculated using the MAS5 software provided by Affymetrix. An expression value is obtained for each gene as well as the indication of the significance of the value obtained. The calculation of the significance of the expression is based on the analysis of the signals, which are obtained following hybridization of the cRNA of a given gene with an oligonucleotide that is a perfect match compared with an oligonucleotide which contains a single mismatch in the central region of the oligonucleotide (see Table 1).

TABLE 1
Measurement of the expression of SCARB-1 in the epidermis and in the
human sebaceous gland by the use of the Affymetrix technology.

| | | | | Significance | Significance |
|---|---|---|---|---|---|
| | | | | of the | of the |
| | | Expression | Expression | expression* | expression* |
| Affymetrix | Name of | in the human | in the human | in the human | in the human |
| identifier | the gene | sebaceous gland | epidermis | sebaceous gland | epidermis |
| 201819_at | Scavenger | 190 | 77 | 1 | 1 |
| | receptor | | | | |
| | class B, | | | | |
| | member 1 | | | | |

*Indicator of the significance of the gene analyzed in the sample indicated: presence (=1) or absence (=0).

Results:

The SCARB-1 receptor is well expressed in both tissues (sebaceous gland, epidermis). Differential analysis from the expression in the human sebaceous gland and the human epidermis shows that SCARB-1 is expressed more highly in the human sebaceous gland.

### Example 2

### Expression of SCARB-1 in the mouse preputial gland

A. The mouse preputial glands show differentiation of the sebocyte type and are used as an experimental model for a sebaceous gland. They have a sufficient size to allow isolation of RNA without having recourse to microdissection technologies.

Analysis of the expression of SCARB-1 in the mouse preputial glands was carried out under conditions of deficiencies of steroid hormones

(in particular of androgenic hormones) following a gonadectomy. The gonadectomized animals were then treated with physiological quantities of Dihydrotestosterone (DHT) or Dihydroepiandrosterone (DHEA) in order to restore a physiological level of androgenic hormones, or as a control experiment with a DHEA-Flutamide combination in which the Flutamide, an antagonist of the androgen receptors, blocks the effect of DHEA. Comparison of the gene expression under these experimental conditions makes it possible to unambiguously identify the modulation or non-modulation of the gene expression of a gene in question by the androgenic hormones (FIG. 1).

The gene expression was analyzed using the Affymetrix technology described above.

Result:

The mRNA for SCARB-1 is induced by a chronic treatment for 7 days with androgens in the preputial gland.

B. Male mice were gonadectomized and treated with the vehicle or DHT. A kinetic study of the expression of the gene from 15 minutes to 96 hours was carried out. For this, the preputial glands were removed for a period ranging up to 4 days (androgenic treatment alone observation of a short-term kinetics), the RNA was isolated and the expression of the genes was analyzed by the Affymetrix technique (FIG. 2).

Results:

Gonadectomy (which causes a steroid hormone deficiency) induces a reduction in the quantity of mRNA for SCARB-1 in the mouse preputial gland.

The mRNA for SCARB-1 in the mouse preputial gland is not induced by a short-term treatment with DHT (effect not visible at 18, 24, 48 and 96 hours).

Each patent, patent application, publication, text and literature article/report cited or indicated herein is hereby expressly incorporated by reference in its entirety.

While the invention has been described in terms of various specific and preferred embodiments, the skilled artisan will appreciate that various modifications, substitutions, omissions, and changes may be made without departing from the spirit thereof. Accordingly, it is intended that the scope of the present invention be limited solely by the scope of the following claims, including equivalents thereof.

What is claimed is:

1. An in vitro method for screening candidate compounds for the preventive and/or curative treatment of acne, or skin disorders associated with a hyperseborrhea, comprising determining the capacity of a candidate compound to modulate the expression or activity of the SCARB-1 receptor or the expression of its gene or the activity of at least one of its promoters.

2. An in vitro method for screening candidate compounds for the preventive and/or curative treatment of acne and/or skin disorders associated with a hyperseborrhea as defined by claim 1, comprising the following steps:
a. preparing at least two biological samples or reaction mixtures;
b. bringing one of the samples or reaction mixtures into contact with one or more test compounds;
c. measuring the expression or activity of the SCARB-1 receptor, the expression of its gene or the activity of at least one of its promoters, in biological samples or reaction mixtures;
d. selecting the compounds for which a modulation of the expression or activity of the SCARB-1 receptor, or a modulation of the expression of its gene or a modulation of the activity of at least one of its promoters, is measured in the sample or mixture treated in b), compared with the untreated sample or mixture.

3. The in vitro method as defined by claim 2, wherein the compounds selected in step d) inhibit the expression or the activity of the SCARB-1 receptor, or the expression of its gene or the activity of at least one of its promoters.

4. The in vitro method as defined by claim 2, wherein the biological samples are cells transfected with a reporter gene that is operably linked to all or part of the promoter of the gene encoding the SCARB-1 receptor, and in that step c) comprises measuring the expression of the said reporter gene.

5. The in vitro method as defined by claim 2, wherein the biological samples are cells expressing the gene encoding the SCARB-1, and in that step c) comprises measuring the expression of the said gene.

6. The in vitro method as defined by claim 4, wherein the cells are sebocytes.

7. The in vitro method as defined by claim 4, wherein the cells are cells transformed with a heterologous nucleic acid encoding the SCARB-1 receptor.

8. The in vitro method as defined by claim 2, wherein the expression of the gene is determined by measuring the level of transcription of the said gene.

9. The in vitro method as defined by claim 2, wherein the expression of the gene is determined by measuring the level of translation of the said gene.

10. The in vitro method as defined by claim 2, wherein the reaction mixtures each comprise an SCARB-1 receptor, and in that step c) comprises measuring the activity of the receptor.
